(19) United States
(12) Patent Application Publication
(10) Pub. No.: US 2024/0226826 A1
(43) Pub. Date: Jul. 11, 2024

(54) METHOD FOR EXTRACTING PLASMID DNA IN BACTERIA

(71) Applicant: ADVACCINE (SUZHOU) BIOPHARMACEUTICALS CO. LTD., SUZHOU (CN)

(72) Inventors: Hongkai Yang, SUZHOU (CN); Zongsheng Wu, SUZHOU (CN)

(21) Appl. No.: 18/559,309

(22) PCT Filed: Mar. 24, 2022
(86) PCT No.: PCT/CN2022/082698

§ 371 Date: Nov. 6, 2023

(30) Foreign Application Priority Data
May 10, 2021 (CN) 202110505674.7

(51) Int. Cl. B01F 29/25; B01F 33/40; C12N 1/06; C12N 15/10

(52) U.S. Cl. CPC B01F 29/25; B01F 33/404; C12N 1/06; C12N 15/1017

(57) ABSTRACT

Provided is a method for extracting plasmid DNA in bacteria, realizing lysis and neutralization during plasmid production in two mixing assemblies connected in series, and comprising the following steps: (1) mixing, (2) lysing, and (3) neutralizing. Step (1) is completed in a first mixing assembly; step (2) is completed in a lysis helical tube; step (3) is completed in a second mixing assembly; and the first mixing assembly, the lysis helical tube and the second mixing assembly are sequentially connected in series. A device used in the plasmid preparation process is simple, is convenient to operate, is low in costs, can remove a large amount of impurities during cell lysis without professional customized device and expensive device, has safe components, realizes automatic continuous lysis, and facilitates industrial production.

### CROSS-REFERENCE TO RELATED APPLICATIONS

The present disclosure claims the priority of the Chinese patent application No. CN 202110505674.7 entitled “METHOD FOR EXTRACTING PLASMID DNA FROM BACTERIA” filed to the Chinese patent office on May 10, 2021, the entire contents of which are incorporated by reference in the present disclosure.

### TECHNICAL FIELD

The present disclosure belongs to the technical field of biological pharmacy, and particularly relates to a method for extracting plasmid DNA from bacteria.

### BACKGROUND

In recent years, the need for fermentation production of plasmids on an industrial scale has been pressing owing to the clinical success of gene therapy and DNA vaccines. Gene therapy is the introduction of foreign genes into target cells to express genes in patient cells to treat or prevent diseases, with the ultimate goal of curing inherited and acquired diseases by adding, correcting, or replacing genes. There are two main types of gene therapy vectors that achieve these goals, i.e., viral vectors based on inactivated viruses and non-viral vectors based on plasmid DNA. The DNA vaccine is also called nucleic acid vaccine or gene vaccine, and is characterized by that the recombinant eukaryotic expression vector for coding a certain protein antigen can be directly injected into a human body, so that the exogenous gene can be expressed in vivo, and the produced antigen activates the immune system of the human body so as to induce specific humoral immunity and cellular immune response. The DNA vaccine is called a “third generation vaccine” following the inactivated vaccine, the attenuated vaccine and the subunit vaccine, and has a broad development prospect, and the plasmid is a common carrier of the DNA vaccine. Therefore, the technology of mass production of plasmids is crucial for the development of gene therapy and DNA vaccines.

The current large-scale plasmid production technology mainly comprises the following steps: vector construction, bacterial fermentation, bacteria lysis, solid-liquid separation and clarification, and plasmid purification. Although the current plasmid production processes can produce plasmids meeting pharmaceutical quality standards and meet clinical requirements, there are some insurmountable bottlenecks in these processes. For example, the large-scale (kilogram-level) yield is difficult, the problems of carrier copy number and stability exist, DNA denaturation and HCD residue removal are caused in the lysis process, solid-liquid separation is difficult, endotoxin residues exist, and the like.

Plasmid DNA for biological pharmacy is mainly produced in Escherichia coli, the alkaline lysis method is the most widely applied method for preparing plasmid DNA, cells are lysed under alkaline conditions, meanwhile, irreversible denaturation is generated on the chromosome DNA, and the plasmid and the chromosome DNA are separated by the principle that the plasmid DNA can renature when the pH is recovered to be neutral. The first step, also the most critical step, of plasmid preparation is cell lysis, and how to thoroughly lyse cells, completely co-precipitate chromosomal DNA, and remove most of RNAs become the core problem of cell lysis. The main problems of the current industrial scale plasmid extraction process are: 1. there is no automatic equipment for alkaline lysis or the capacity of the equipment cannot meet the requirement of full mixing; 2. a large number of organic solvents and acid liquor are used, which increases the safety risk in large-scale industrial production and has higher requirements on plant equipment; 3. after mixing using the neutralization solution (solution III), the mixing equipment without automation or the mixing equipment cannot meet the mixing requirement of uniform low shear; 4. the production cost for solid-liquid separation of the neutralized mixed liquid is high; 5. the residual host DNA is high; 6. the RNA removal rate is low; and downstream purification is influenced; 7. it is difficult to wash pipeline equipment that is reused, which is unfavorable for CIP washing, and disposable consumptive material equipment cost is high.

The Chinese patent CN111808716A discloses a plasmid extraction device, including a lysis container, a precipitation container, an eluent container, a collecting container and a chromatographic column, the lysis container is communicated with the precipitation container through a first connecting pipe, the precipitation container is communicated with the precipitation container through a second connecting pipe, the eluent container is communicated with the chromatographic column through a third connecting pipe, the first connecting pipe, the second connecting pipe and the third connecting pipe are all provided with a valve, the collecting container is provided below the chromatographic column, and the chromatographic column is connected with a vibration mechanism. The technical solution above adopts a vibrating structure, the processing process is discontinuous, the processing efficiency needs to be further improved, and the method has the problem of high residual host DNA and needs to be further improved.

Although we have previously developed a method for extracting plasmid DNA from bacteria by lysis and neutralization in a mixing cavity oscillation manner (see: 200610114061.6, a method for continuously extracting a large number of plasmids) in order to solve the defects existing in the current method for large-scale production of plasmid DNA, scaling up is not easy and the efficiency of extracting plasmid DNA is low; to solve this problem, we have further developed a device for extracting plasmid DNA by means of a bubble mixer (see: 202011120617.9, a bubble generating device for extracting plasmid DNA from bacteria), which allows the bacterium solution and the lysate to be uniformly and sufficiently mixed, and the bubble mixing effectively reduces the shear force and effectively improves the yield and quality, but which yet still has the problem of being not easily scaled up; and it is necessary to customize bubble mixers of different sizes according to the scale, and to search for the scaling conditions such as the amount of ventilation and the flow rate.

Therefore, we also disclose and design a novel method for extracting plasmid DNA, which can control the shear force of the mixing process effectively and achieve the scale-up process more easily. Moreover, compared with the bubble mixer (bubble generating device) method, the new method can effectively improve the mixing efficiency in the lysis and neutralization process and increase the yield; and by easily and controllably adjusting the mixing parameters, the shear force can be effectively controlled and the plasmid quality is improved. Furthermore, the device used in the process has a simple principle, and can be accurately regulated, so that the time for exploring the scale-up conditions is shortened, the working efficiency is further improved, the development of related researches on continuous and large-scale extraction of plasmid DNA can be greatly promoted, and the method is of great significance.

### SUMMARY

Regarding the problems in the prior art, the present disclosure provides a method for extracting plasmid DNA from bacteria, which requires simple equipment, is easy to operate, has a low cost, requires no professional customized and expensive equipment, is capable of removing a large number of impurities in the cell lysis process, involves safe components, and can realize automatic continuous lysis, and facilitates industrial production.

In one aspect, the present disclosure provides a method for extracting plasmid DNA from bacteria, wherein lysis and neutralization in plasmid production process are implemented in two mixing assemblies connected in series, specifically comprising the following steps:
(1) mixing;
(2) lysing;
(3) neutralizing;
   wherein step (1) is completed in a first mixing assembly, step (2) is completed in a spiral lysis pipe, step (3) is completed in a second mixing assembly, and the first mixing assembly, the spiral lysis pipe, and the second mixing assembly are sequentially connected in series. Preferably, the first mixing assembly has a rotational speed of 50 rpm to 1,500 rpm, preferably 200 rpm to 500 rpm; the second mixing assembly has a rotational speed of 20 rpm to 1,000 rpm, preferably 150 rpm to 500 rpm.

In some embodiments, in step (2), the lysis is performed for 2 min to 10 min, preferably 5 min.

In some embodiments, the structures of the first mixing assembly and the second mixing assembly are each independently selected from any one of a stirring type, an emulsifying type and a centrifugal type, and the first mixing assembly and the second mixing assembly are both preferably mixing pumps or stirrers; preferably, the first mixing assembly is of the stirring type or the emulsifying type or the centrifugal type, and the second mixing assembly is of the centrifugal type.

In some embodiments, the first mixing assembly and the second mixing assembly are a first mixing pump and a second mixing pump respectively, and the ratios of the pump cavity volume of the first mixing pump and the pump cavity volume of the second mixing pump to the rated feed volume per minute of a single mixing pump are both in the range of 1:6-1:1, preferably 1:6-1:3; or the volumes of the pump cavities of the first mixing pump and the second mixing pump are both the volume of the feed liquid flowing through the pump cavity for 10 s to 60 s, preferably the volume of the feed liquid flowing through the pump cavity for 10 s to 20 s. Impellers of the first mixing pump and the second mixing pump are both preferably semi-enclosed impellers. By adopting a mixing pump, the lysis and neutralization processes are in an airtight environment, which reduces the probability of polluting the environment and facilitates CIP and SIP after use.

In some embodiments, the spiral lysis pipe has an inner diameter of 0.5 cm to 15 cm, preferably 0.5 cm to 6 cm; pump heads of the first mixing pump and the second mixing pump both have a diameter of 2 cm to 100 cm, preferably 4 cm to 30 cm.

In some embodiments, impellers of the first mixing pump and the second mixing pump both comprise a rear cover plate; a plurality of flow guide columns are uniformly distributed on the rear cover plate, and the outer side surface at least along the rotating direction of the impeller on the flow guide column is arranged as an arc surface.

In some embodiments, the flow guide column is a cylinder, a circular truncated cone, a fan-shaped column, or a combination of one or more thereof.

In a typical embodiment, the cross section of the flow guide column has a width of 0.5 mm to 40 mm, preferably 2 mm to 10 mm. That a plurality of flow guide columns are uniformly distributed and the diameters thereof are within suitable ranges can reduce the shear force, prevent the host DNA from polluting products, and enables lysis and neutralization to be carried out automatically.

In a typical embodiment, the flow guide column is preferably a cylinder, or the cross-sectional area of the middle of the flow guide column is the largest, and the cross-sectional areas from the middle to the two ends gradually decrease, and in a specific implementation, the flow guide column may have a spindle shape.

In some embodiments, the plasmid preparation process specifically comprises the following steps:
(1) resuspending the bacteria by using the solution I to obtain a resuspended bacterium solution, and then introducing the resuspended bacterium solution and solution II into the first mixing assembly for mixing to obtain a bacteria mixed solution;
(2) the bacteria mixed solution flowing out of the first mixing assembly and entering the spiral lysis pipe for lysis, to obtain a lysate;
(3) introducing the lysate and solution III into the second mixing assembly and performing a neutralization reaction (or mixing the lysate and the solution III before introducing them into the second mixing assembly) to obtain a neutralization reaction solution after the neutralization reaction is finished;
   preferably, after obtaining the neutralization reaction solution, the method further comprising a step of performing solid-liquid separation and purification on the neutralization reaction solution.
   wherein
   in step (1), the volume to mass ratio of the resuspended bacterium solution to the bacteria is 3-20:1 (L:kg), further preferably 7:1 (L:kg).
   In step (1), the volume ratio of the solution I to the solution II is 1:0.5-1:3, further preferably 1:1. The alkali lysis time is controlled to be 2 min to 10 min through different pipeline thicknesses and lengths, thus ensuring complete bacteria lysis and the lysis effect.

In step (1), the solution I comprises Tris-HCl and EDTA-2Na, and further preferably, the Tris-HCl has a concentration of 2 mmol/L to 100 mmol/L, the EDTA-2Na has a concentration of 0.1 mmol/L to 50 mmol/L, and the solution I has a pH in the range of 6.0 to 9.0.

In step (1), the solution II comprises NaOH and SDS, and further preferably, the NaOH has a concentration of 0.02 to 5 mol/L, and the SDS has a concentration of 0.1-10%.

In step (2), the lysis time is 2 min to 10 min, further preferably 5 min.

In step (3), the solution III comprises KAc and NH₄Ac, and further preferably, the KAc has a concentration of 0.1 mol/L to 6 mol/L, and the NH₄Ac has a concentration of 0.2 mol/L to 10 mol/L.

In step (3), the volume ratio of the lysate to the solution III is 1:0.3-5, further preferably 1:1. The lysis and neutralization effects are controlled by the above conditions, and the precipitation of host DNA and the removal effect of host RNA are ensured.

In some embodiments, the solid-liquid separation is carried out by a filtration assembly, and the solid-liquid separation method includes but is not limited to one or a combination of filtration, depth filtration, centrifugation, and the like.

In some embodiments, the solid-liquid separation is carried out by a filtration assembly, wherein the structure of the filtration assembly is a sieve type, a depth filtration type, a centrifugal filtration type, or a combination of one or more thereof; further preferably, the filtration assembly has a structure of a sieve or depth filtration type; the pore size of the filter is 0.2 μm to 800 μm, preferably 0.1 μm to 200 μm; the filter material includes cellulose, diatomite, activated carbon, polypropylene fiber and silica gel.

In some embodiments, the filter material includes, but is not limited to, one or a combination of cellulose, diatomite, activated carbon, polypropylene fiber, silica gel, polyethersulfone, nylon, and polyvinylidene fluoride.

In some embodiments, the filtration assembly structure is a centrifugal structure; the centrifugal force is 1,000 g to 20,000 g, the centrifugal time is 2 min to 60 min, and the temperature is 2° C. to 40° C.

In another aspect, the present disclosure also provides a device for extracting plasmid DNA from bacteria by the above method, comprising: a first mixing assembly and a second mixing assembly;
the first mixing assembly and the second mixing assembly are connected through the spiral lysis pipe; at least one liquid inlet is provided on a connecting pipeline between the spiral lysis pipe and the second mixing assembly;
after the resuspended bacterium solution flows into the first mixing assembly for mixing, lysis is carried out through the spiral lysis pipe to obtain a lysate, then the lysate is introduced into the second mixing assembly to be neutralized with the solution III to obtain a neutralization reaction solution, and the lysate enters into the second mixing assembly through the liquid inlet.

In some embodiments, the spiral lysis pipe has an inner diameter of 0.5 cm to 15 cm, preferably 0.5 cm to 6 cm; pump heads of the first mixing pump and the second mixing pump both may have a diameter of 2 cm to 100 cm, preferably 4 cm to 30 cm.

In some embodiments, the length of the flow guide column is related to the distribution position, the length of each flow guide column decreases sequentially from the center of the rear cover plate to the outer edge, and the vertexes of the flow guide columns are located on the same paraboloid.

In some embodiments, the liquid inlet ends of the first mixing pump and the second mixing pump can be coaxially disposed with the liquid outlet ends; the liquid inlet end is positioned at the center of the pump housing, and the liquid outlet end is positioned at the center of the pump base. Therefore, the fluid entering into the pump cavity can be discharged only after passing sequentially from the center to the edge of the rear cover plate and winding to the rear, so that the fluid is fully contacted with the flow guide column, the object of uniform mixing is achieved, and the neutralization reaction quality is improved.

In some embodiments, the device further comprises a filtration assembly, the liquid outlet end of the second mixing assembly is connected to the liquid inlet end of the filtration assembly, and the neutralization reaction solution is filtered by the filtration assembly.

In some embodiments, the resuspended bacterium solution comprises solution I and bacteria containing plasmid DNA, and the resuspended bacterium solution is mixed and conveyed to the first mixing assembly by a first conveying pump, mixed with solution II conveyed to the first mixing assembly by the second conveying pump, and then introduced into the spiral lysis pipe for lysis.

Compared with the prior art, the beneficial effects of the present disclosure are:
(1) The mixing assembly (which can be a pump) is innovatively adopted in the alkaline lysis and neutralization step in the plasmid production process of the present disclosure, so that the lysis and neutralization steps are performed in an airtight environment, the probability of environmental pollution is reduced, CIP and SIP can be conveniently performed after use, continuous processing is realized, the production efficiency is improved, the cost is low; professional customized and expensive equipment is not needed, the scale-up in production is easy, and the production cost is low; the mixing is sufficient during lysis, the mixing time is short, the neutralization condition is mild and uniform, the residual host DNA and RNA are lower than that of a foaming mixer after the lysis and neutralization, and the product quality is good; meanwhile, the size of the pump cavity is optimized, so that the time and the shear force for the lysis and neutralization are suitable for product production, the production scale is convenient to be scaled-up, is easily scaled-up compared with the production system of the current mainstream bubble mixer Airmix, the bubble mixers with different sizes are not required to be customized according to the scale, the exploration time of the scale-up condition is shortened, and the working efficiency is improved.
(2) In the method for extracting plasmid DNA, the equipment used is simple, the operation is convenient, the two mixing assemblies used can fully mix the bacterium solution and the lysate and ensure that the neutralization solution is mildly mixed and neutralized, the complex low-shear neutralization equipment is avoided, the superhelix proportion of the plasmid after lysis is high, and the residual host DNA and RNA are low; in addition, by using a complex multi-level membrane filtration system, overnight precipitation and other steps are not needed after lysis, the equipment can be directly cleaned by CIP so as to meet the production specification of drug production, meanwhile, the process time is saved, and the cost is reduced; a complex multi-level membrane filtration system is not used, overnight precipitation and other steps are not needed after lysis, the superhelix proportion of the plasmid after lysis is high, the residual host DNA and RNA are low; the equipment can be directly cleaned by CIP so as to meet the production specification of drug production, meanwhile, the process time is saved, the cost is reduced, the operation is convenient, professional customized and expensive equipment is not needed, the scale-up in production is easy, and the production cost is low.
(3) In the extraction process of the plasmid DNA, high-risk animal source components such as RNase, lysozyme, proteinase K and the like are not added, toxic organic solvents such as isopropanol, phenol, absolute ethyl alcohol, other mutagens and the like are not used in the production process, the reagents used can be common reagents or of the medicinal grade, acid liquor is not used for neutralization, the requirement on plant equipment is low, and the method is suitable for large-scale production.
(4) By optimizing the size of the mixing pump cavity and adjusting the ratio of the pump cavity to the flow rate, the time and shear force for the lysis and neutralization are suitable for product production, and meanwhile, the scale-up of production is also facilitated; by optimizing the property and size of the mixing pump head, using 3D printing technique, and designing and customizing the pump head and in the case that the mixing effect is ensured, the shear force is reduced, host DNA is prevented from polluting products, and lysis and neutralization can be performed automatically.

### BRIEF DESCRIPTION OF THE DRAWINGS

FIG. 1 is a schematic view of a device for extracting plasmid DNA from bacteria according to the present disclosure.

FIG. 2 is a stereoscopic view of a first mixing assembly impeller of a device according to a example of the present disclosure.

FIG. 3 is a stereoscopic view of a second mixing assembly of a device according to a example of the present disclosure, wherein the arrows indicate feed liquid flow directions.

FIG. 4 is an exploded view of the second mixing assembly in FIG. 3.

FIG. 5 is a structural view of the second mixing assembly in FIG. 3 with the pump housing being removed.

FIG. 6 is a stereoscopic view of the second mixing assembly impeller in FIG. 3.

FIG. 7 is a diagram showing a comparison of electrophoresis results in Example 1, wherein lane 1 is a marker, lane 2 is a supernatant of the lysis and neutralization reaction solution in Example 1, and lane 3 is a standard.

FIG. 8 is a diagram showing a comparison of electrophoresis results in Example 1, wherein lane 1 is a supernatant of the neutralization reaction solution in Example 1, lane is a supernatant of the neutralization reaction solution in Comparative Example 1, lane 3 is a standard, and lane 4 is a marker.

FIG. 9 is a schematic view of the impeller used in Comparative Example 2.

FIG. 10 is a schematic view of the impeller used in Comparative Example 3.

FIG. 11 is a diagram showing a comparison of electrophoresis results in Comparative Example 3, wherein lane 1 is a supernatant of the neutralization reaction solution in Example 1, lane 2 is a supernatant of the neutralization reaction solution in Comparative Example 3, lane 3 is a standard, and lane 4 is a marker.

FIG. 12 is a diagram showing a comparison of electrophoresis results in Examples 1, 2 and 3, wherein lane 1 is a supernatant of the neutralization reaction solution in Example 3, lane 2 is a supernatant of the neutralization reaction solution in Example 1, lane 3 is a supernatant of the neutralization reaction solution in Example 2, lane 4 is a standard, and lane 5 is a marker.

FIG. 13 is a schematic view of a three-dimensional structure of the flow guide column in Example 4; in FIGS. 1-6 shown above:

1—first mixing assembly; 2—second mixing assembly; 201—main shaft; 202—pump base; 203—sealing ring; 204—impeller; 205—pump housing; 2021—annular groove; 2041—rear cover plate; 2042—flow guide column; 3—spiral lysis pipe; 4—filtration assembly; 5—resuspended bacterium solution; 6—solution II; 7—solution III.

### DETAILED DESCRIPTION

The following non-limiting examples may enable those of ordinary skill in the art to understand the present disclosure more comprehensively, but are not intended to limit the present disclosure in any way. The following is merely an illustrative description of the scope claimed by this application, and various changes and modifications can be made in the disclosure of this application by those skilled in the art based on the content disclosed, which also fall within the scope claimed in this application. The drawings described below are merely one or several examples of the present disclosure, and other drawings may be derived by those of ordinary skill in the art without making creative efforts.

The present disclosure is further illustrated by the following specific examples. The various chemicals used in the examples of the present disclosure are available from conventional commercial sources unless otherwise specified. Unless otherwise specified, the concentration percentage is a mass percentage.

Unless otherwise indicated, the term “lysis solution” herein refers to “solution II”. Unless otherwise indicated, the “neutralization solution” herein refers to “solution III”. In the following examples, the next purification step may be performed by conventional purification means in the art.

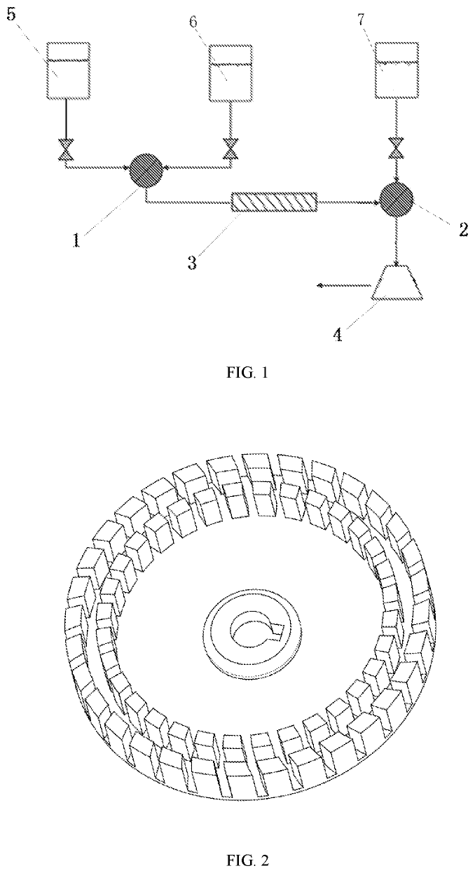

The device used in the present disclosure:
as shown in FIG. 1, mainly comprising a first mixing assembly 1, a second mixing assembly 2, and a filtration assembly 4. The two mixing assemblies are distinguished by function, the first mixing assembly 1 can be a lysis mixing assembly, and the second mixing assembly 2 is a neutralization mixing assembly.

Preferably, the first mixing assembly 1 is connected in series with the second mixing assembly 2; a spiral lysis pipe 3 is also connected in series between the two mixing assemblies. Specifically, the solution I and the bacteria containing plasmid DNA were proportioned to form a resuspended bacterium solution 5, the resuspended bacterium solution 5 is conveyed to the first mixing assembly 1, namely the lysis mixing pump, and the flow and the flow speed are controlled by a first conveying pump. In a specific implementation, a first three-way joint (namely a Y-shaped connector) is connected in series on the conveying pipeline, and the resuspended bacterium solution and the solution II 6 are respectively conveyed to the first three-way joint through the first conveying pump and a second conveying pump and then introduced into the first mixing assembly 1 for mixing to obtain a bacteria mixed solution.

The liquid outlet end of the first mixing assembly 1 is connected with the liquid inlet end of the spiral lysis pipe 3; the liquid outlet end of the spiral lysis pipe 3 was connected with the liquid inlet end of the second mixing assembly 2. A liquid inlet, in particular a second three-way joint in the pipeline connected in series, is arranged on the pipeline between the spiral lysis pipe 3 and the second mixing assembly 2, and one end of the second three-way joint is also connected with a container of the solution III 7 through a third conveying pump.

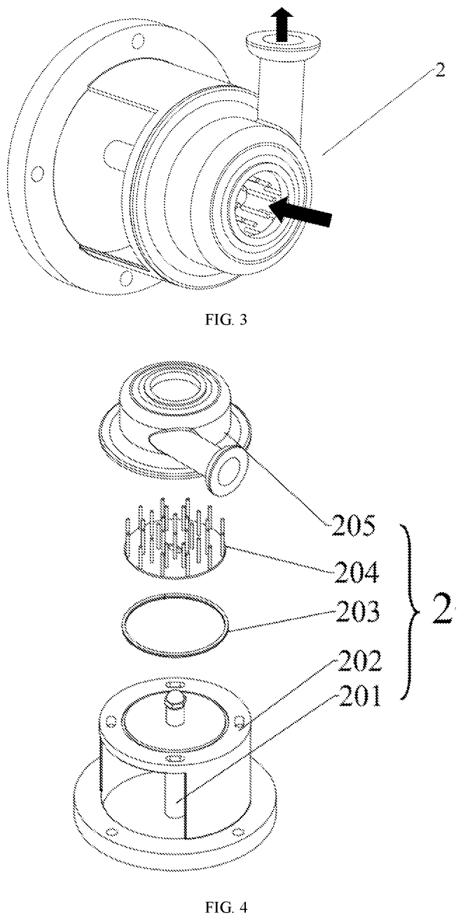

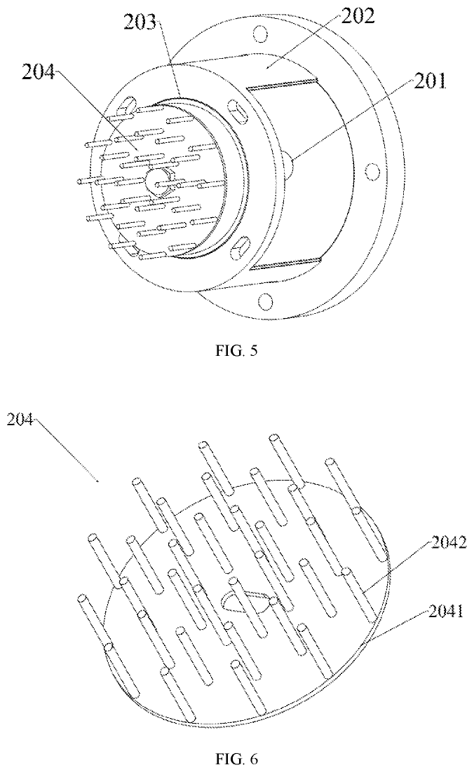

The first mixing assembly 1 and the second mixing assembly 2 used in this example may be a stirrer or a mixing pump, and specifically may be a first mixing pump and a second mixing pump, respectively, including but not limited to a stirring pump, an emulsification pump, a centrifugal pump, and the like, wherein a stirring blade of the stirring pump may be selected from a paddle stirrer, a propeller stirrer, a turbine stirrer, an anchor stirrer, a frame stirrer, and a screw stirrer; the rotor and stator of the emulsification pump include, but are not limited to: coarse teeth, middle teeth and fine teeth. Through the pump head of a certain regular shape, full mixing of the solution is achieved and the shear force is low; ensuring that chromosome DNA does not involve a large amount of fracture, and can be carried out under an airtight environment without causing pollution. Further, because the first mixing assembly 1 is used for the lysis reaction, the structure of the first mixing assembly 1 is preferably selected to be a stirring type or an emulsifying type, and specifically, an emulsification pump can be selected. The structure of the blade (or impeller) thereof is shown in FIG. 2 (it can also be in the form of other emulsification pumps in the prior art, and is only shown in FIG. 2 herein);
the second mixing assembly 2 is used for neutralization reaction, and can be a centrifugal structure, and as shown in FIG. 3 and FIG. 4, the second mixing assembly 2 mainly includes a main shaft 201, a pump base 202, a sealing ring 203, an impeller 204, and a pump housing 205. The fluid conveying route is as shown by the arrows in FIG. 3. The fluid enters into the pump through the liquid inlet end at the center of the pump housing 205. After centrifugal mixing, the fluid can flow out through the liquid outlet end arranged laterally on the pump housing 205, and the pipeline in the liquid outlet end is tangent to the inner cavity of the pump. One end of the main shaft 201 is connected with the output end of an external motor, the other end of the main shaft 201 penetrates through the center of the pump base 202 through a sealing device and is fixedly connected with the impeller 204, and an annular groove is processed in the contact area of the pump base 202 and the pump housing 205 and is used for mounting the sealing ring 203. The impeller 204 is preferably a semi-enclosed impeller; however, the conventional impeller has a great disadvantage that the shear force is great. Therefore, in this example, the impeller 204 is designed as shown in FIG. 5 and FIG. 6, comprising a rear cover plate 2041. A plurality of flow guide columns 2042, 32 in total, are uniformly distributed on the rear cover plate 2041, and surround the center in three layers, and the flow guide columns 2042 are perpendicular to the surface of the rear cover plate 2041. Further, in order to better reduce the generated shear force, the shape of the flow guide column 2042 may be one or a combination of a cylinder, a circular truncated cone, or a fan-shaped column, preferably a cylinder. The flow guide column 2042 has a diameter of 0.5 mm to 40 mm; as proved by tests, better effects can be achieved when the diameter is preferably 2 mm to 10 mm. Through optimal design, the shear force can be reduced, the host DNA is prevented from polluting the product, and the lysis and neutralization can be automatically carried out. Experiments show that the second mixing assembly 2 controls the mixing effect and the shear force of different scales by setting a certain rotational speed range, and automatic lysis and neutralization of bacterium solution of different scales can be realized by combining the first mixing assembly 1, thereby realizing continuous and large-scale production.

In specific examples, the structure of the first mixing assembly 1 may also preferably be identical to the structure of the second mixing assembly 1.

Specifically, good mixing effects are achieved when the impeller rotational speed of the second mixing assembly 2 is 20 rpm to 1000 rpm. The pump head property, size, and rotational speed can also be changed for the second mixing assembly 2 to control the neutralization effect; the second mixing pump has a pump head diameter of 2 cm to 100 cm, preferably 4 cm to 30 cm, the rotational speed is controlled at 20 rpm to 1000 rpm, preferably 150 rpm to 500 rpm, and the ratio of the pump cavity volume to the rated feed volume per minute of the mixing pump is in the range of 1:6-1:1, preferably 1:6-1:3; or the volume of the pump cavity is designed to be the volume of the feed liquid flowing through the pump cavity for 10 s to 60 s, preferably the volume of the feed liquid flowing through the pump cavity for 10 s to 20 s, so that complete neutralization is ensured, lower shear force is generated, the breakage of chromosome DNA is reduced, and the quality of plasmid DNA is improved.

The liquid outlet end of the second mixing assembly 2 is connected with the liquid inlet end of the filtration assembly 4.

Preferably, the filtration assembly 4 is one or a combination of a sieve type, a depth filtration type and a centrifugal filtration type. Specifically, the structure of the filtration assembly 4 in this example is a structure of a depth filtration type; the pore size of the filter well is 0.2 μm to 800 μm; the specific optional pore size of the filter well can be between 0.1 μm and 200 μm. Second clarification is performed on the neutralized supernatant by a depth filtration method, wherein the material component for filtration includes but is not limited to cellulose, diatomite, activated carbon, polypropylene fiber, silica gel and a combination product thereof. The membrane area of the depth filtration membrane is between 0.01 m² and 2 m².

The extraction device in the above examples can all be used in the following examples, where differences exist, they will be shown; the specific method for extracting plasmid DNA from bacteria are as follows:

### Example 1. 50 L Fermentation Scale Processing

In the device for extracting plasmid DNA from bacteria in Example 1, the pump head had a diameter of 10 cm, the pump head impellers of two pumps were both shown in FIG. 6, and the flow guide column had a diameter of 5 mm.
(1) The high density Escherichia coli fermentation bacterium solution containing plasmid A was measured by a spectrophotometer to obtain OD600 of 84.2, and the fermentation broth was centrifuged at 23.3 L to obtain 3684 g of bacteria, with a wet weight of 15.8%. 3684 g of cells were resuspended in a pH 8.0 resuspended solution (solution I) composed of 25 mM Tris-HCl and 10 mM EDTA-2Na to obtain a resuspended bacterium solution with a volume of 25.8 L (the mass to volume ratio of bacteria to the solution I was 1:7 (kg:L)).
(2) The resuspended bacterium solution was pumped at 140 mL/min to one side of the Y-shaped connector, while a lysis solution (solution II) consisting of 0.2 M NaOH and 1% SDS was pumped at 140 mL/min to the other side of the Y-shaped connector. The Y-shaped connector was connected with a lysis mixing pump (a first mixing pump), the rotational speed was adjusted to 200 rpm, and lysis and mixing were initiated to obtain a bacteria mixed solution. The volume ratio of the solution I to the solution II is 1:1, and the pump cavity volume of the lysis mixing pump is 1:3 of the rated feed volume per minute of a single mixing pump.
(3) After being pumped out of the first mixing assembly (lysis mixing pump), the bacteria mixed solution entered into the spiral lysis pipe, wherein the spiral lysis pipe had an inner diameter of 1.9 cm and a length of 5 m, and the lysis time in the lysis spiral pipe was 5 min, so as to obtain a lysate.
(4) The lysate after lysis entered into another Y-shaped connector, and the solution III (pre-cooled 2-8° C.) composed of 1 M KAc and 7 M NH₄Ac at the other end of the connector entered at a speed of 280 mL/min, passed through the Y-shaped connector and entered into the neutralization mixing pump (second mixing assembly), wherein a rotational speed of 250 rpm was set for the mixing pump. The volume ratio of the lysate to the solution III was 1:1. The flow guide column on the impeller of the neutralization mixing pump had a diameter of 5 mm, the flow guide column was a cylinder, and the neutralization mixing pump had a pump head diameter of 8.5 cm; wherein the pump cavity volume of the neutralization mixing pump was 1:4 of the rated feed volume per minute of the single mixing pump.
(5) After neutralization was completed, the neutralization reaction solution was collected and centrifuged for 20 min at a centrifugal force of 8000 g, and a supernatant was collected and a further purification step can be performed.

### Test Results:

The plasmid concentration of the resuspended bacterium solution was measured to be 545 mg/L (determined by a plasmid mini kit of QIAGEN) and the total amount of the plasmids was 14.06 g.

100 L of neutralization reaction solution was obtained after neutralization, 82 L of a supernatant was obtained by centrifugation in total, and the plasmid concentration of the supernatant was measured to be 121.8 mg/L by HPLC quantification method (HPLC model: Waters 2695, chromatographic column model: TOSOH, Tskgel DNA-NPR 4.6 mm×7.5 cm 2.5 μm, the conditions of HPLC determination in the following examples were the same), and the lysis yield was 71%.

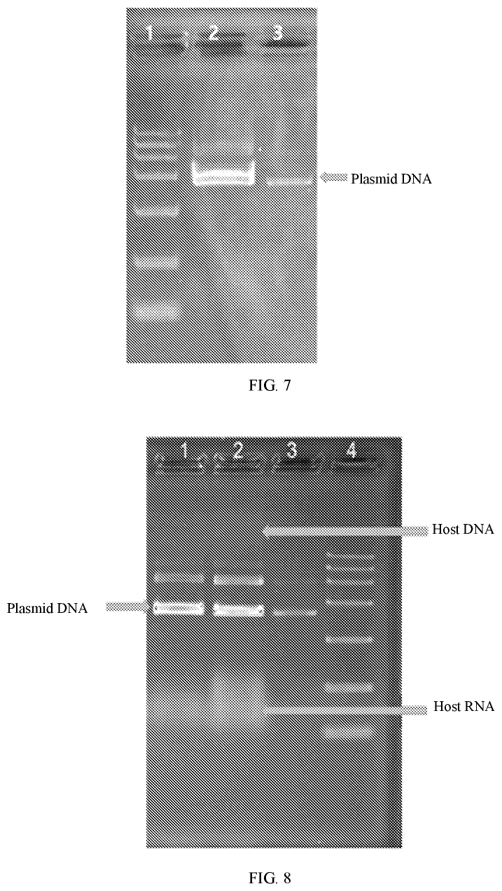

The electrophoresis results were shown in FIG. 7, and it can be seen from FIG. 7 that the plasmid purity in the supernatant after lysis by the method of this application was high, and that the amount of RNA and host DNA was small.

The plasmid DNA prepared by the above method was detected by HPLC tests and pharmacopeia methods, and the results showed that the plasmid was a target plasmid, the purity was high, the superhelix proportion was more than 95%, and the open loop proportion was small.

### Example 2

Unlike Example 1, the rotational speed of the first mixing pump was 400 rpm, and the rotational speed of the second mixing pump was 500 rpm. The flow guide column was a cylinder, and had a diameter of 1 mm. The rest were the same.

Then, the neutralization reaction solution was subjected to agarose nucleic acid electrophoresis, and the electrophoretogram was as shown in FIG. 12. It can be seen that the host DNA and RNA in the lysis supernatant in Example 2 were both higher than those in the lysis supernatant in Example 1, and more impurities were generated at a higher rotational speed.

### Example 3

Unlike Example 1, the resuspended bacterium solution was 2.5 L, the rotational speed of the first mixing pump was 100 rpm, the rotational speed of the second mixing pump was 50 rpm, and the flow guide column was a cylinder with a diameter of 1 mm. The rest were the same.

After neutralization was detected by HPLC, 10 L of the neutralization reaction solution was obtained, 7.8 L of a supernatant was obtained by centrifugation in total, the plasmid concentration of the supernatant was measured to be 96 mg/L (determined by HPLC), and the lysis yield was 55.0%.

The electrophoretogram was shown in FIG. 12, and it can be seen from FIG. 12 that the lower rotational speed of the mixing pump resulted in insufficient mixing and neutralization, and the yield of plasmid DNA was lower than that of Example 1.

### Example 4

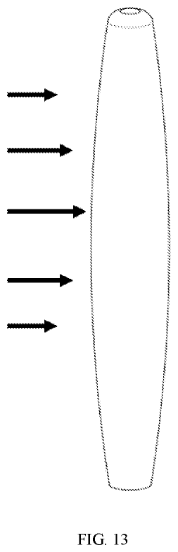

Unlike Example 1, in this example, the flow guide column 2042 was designed as a variable cross-section for the purpose of further reducing the influence of shear on the neutralizing process. Through fluid motion analysis, the arrows in FIG. 13 showed the flow velocity distribution of the fluid relative to the main body in the rotation of a single flow guide column, that is, the velocity was reduced from the middle layer to two sides. The reason was analyzed as that the upper side and the lower side of the fluid were respectively subjected to viscous resistance of a pump housing, namely a pump base, and the speed was distributed in a gradient manner, so that the shear force generated by genetic materials in the fluid by a single flow guide column was kept consistent, and therefore the flow guide column was designed as a variable cross-section structure. Specifically, taking the cylinder in Example 1 as an example, the cross-section of a single flow guide column sequentially increased and then decreased from the pump housing side to the pump base side, so as to form a “spindle-shaped” structure, specifically referring to FIG. 13. For the above-mentioned design, though the flow guide column had a relatively great velocity and impacts strongly at the center thereof, by combining a great radius of curvature and a stressed area, the shear effect on the plasmid can be effectively reduced, thus improving the plasmid yield to a certain extent.

### Example 5

Like Example 1, the result of detecting the host DNA residue (HCD) was 5.87 μg/mg (E. coli residual DNA detection kit). As shown in Table 1, the plasmid DNA was detected by HPLC tests and pharmacopeia methods, and the result showed that the plasmid was a target plasmid, the purity was high, the superhelix proportion was 95.92%, and the open loop proportion was small.

Table 1 shows the HPLC peak results of the sample plasmids and purity detection in Example 5

| | Sample name | | | | |
|---|---|---|---|---|---|
| | Impurity | Open | Super- | Linear, | Impurity |
| | 1, % | loop, % | helix, % | % | 2, % |
| After | ND | 1.15 | 95.92 | ND | 2.93 |
| centrifugation | | | | | |

Note:
“ND” means not detected.
Impurity 1 and impurity 2 are unknown states of the plasmid.

### Example 6

Like Example 2, the result of further detecting the host DNA residue (HCD) was 18.7 μg/mg (E. coli residual DNA detection kit).

### Example 7

Unlike Example 1, in this example, after the lysis and neutralization, the solid-liquid separation was performed by adopting filter bag filtration and depth filtration methods. The purpose was to increase the treatment capacity and improve the production efficiency in the process of scale-up production, reduce the mechanical shear action of a continuous centrifugal machine in production, and reduce the generation of impurities and the damage of target plasmids. Filter bags with a filter area of 0.5 m² and made of polypropylene having a pore size of 100 μm and 200 μm were used for primary filtration, and then the depth filtration with a pore size of the filter well of 0.2 μm to 2 μm and made of a composite material of cellulose and inorganic filter aid was adopted for secondary filtration. The impurity removal rate after the primary filtration can reach 86.2%, the turbidities before and after the filtration was 43 NTU and 10.7 NTU respectively, and the plasmid purity after the filtration was not obviously changed. After secondary filtration, the filtrate was clearer, the turbidity can be reduced to no more than 3 NTU, and downstream purification can be directly carried out. As shown in Table 2, the plasmid DNA was detected by HPLC, and the result showed that the plasmid was a target plasmid, the purity was high, the superhelix proportion reached 96.08% before filtration, the superhelix proportion was 97.6% after filtration with a 100 μm filter bag, and the superhelix proportion was 97.55% after filtration with a 200 μm filter bag.

Table 2 shows HPLC detection results of plasmid purities in clear liquid before and after filtration with 100 μm and 200 μm filter bags

| | Sample name | | | | |
|---|---|---|---|---|---|
| | | | | | Impu- |
| | Impurity | Open | Super- | Linear, | rity |
| | 1, % | loop, % | helix, % | % | 2, % |
| Before filtration | 0.59 | 0.74 | 96.08 | ND | 2.59 |
| After 100 μm filtration | ND | 0.28 | 97.60 | ND | 2.12 |
| After 200 μm filtration | ND | 0.34 | 97.55 | ND | 2.11 |

Note:
“ND” means not detected.
The samples were centrifuged before HPLC sampling and the supernatant was injected.

### Comparative Example 1. Bubble Mixer Treatment

Unlike Example 1, the neutralization step of Comparative Example 1 was carried out in a bubble mixer without using a pump, and the specific steps were as follows:
(1) The high density Escherichia coli fermentation bacterium solution containing plasmid A was measured by a spectrophotometer to obtain OD600 of 78.9. The fermentation broth was centrifuged at 23.5 L to obtain 3603 g of bacteria, with a wet weight of 15.3%. 3603 g of cells were resuspended in a pH 8.0 resuspended solution (solution I) composed of 25 mM Tris-HCl and 10 mM EDTA-2Na to obtain a resuspended bacterium solution with a volume of 25.2 L (the mass to volume ratio of bacteria to the solution I was 1:7).
(2) The resuspended bacterium solution was pumped at 140 mL/min to one side of the Y-shaped connector, while a lysis solution (solution II) consisting of 0.2 M NaOH and 1% SDS was pumped at 140 mL/min to the other side of the Y-shaped connector. The Y-shaped connector was connected with a lysis mixing pump (a first mixing pump), the rotational speed was adjusted to 200 rpm, and lysis and mixing were initiated to obtain a bacteria mixed solution. The volume ratio of the solution I to the solution II is 1:1.
(3) After being pumped out of the lysis mixing pump, the bacteria mixed solution entered into the spiral lysis pipe, wherein the spiral lysis pipe had an inner diameter of 1.9 cm and a length of 5 m, and the lysis time in the lysis spiral pipe was 5 min, so as to obtain a lysate.
(4) The lysate after lysis entered into another Y-shaped connector, and the solution III (pre-cooled 2-8° C.) composed of 1 M KAc and 7 M NH₄Ac at the other end of the connector entered at a speed of 280 mL/min, passed through the Y-shaped connector and entered into the bubble mixer, wherein a compressed air flow rate of 1.2 L/min was set for the bubble mixer. The volume ratio of the lysate to the solution III was 1:1.
(5) After neutralization was completed, the neutralization reaction solution was collected and centrifuged for 20 min at a centrifugal force of 8000 g, and a comparative supernatant was collected and a further purification step can be performed.

The detection by a microplate reader showed that the plasmid concentration of the resuspended bacterium solution was measured to be 570 mg/L (calculated by a plasmid mini kit of QIAGEN) and the total amount of the plasmids was 14.36 g.

After neutralization, 101 L of the neutralization reaction solution was obtained, 79.3 L of a supernatant was obtained by centrifugation in total, the plasmid concentration of the comparative supernatant was measured to be 116.3 mg/L (determined by HPLC), and the lysis yield was 64.2%, which was lower than that of Example 1.

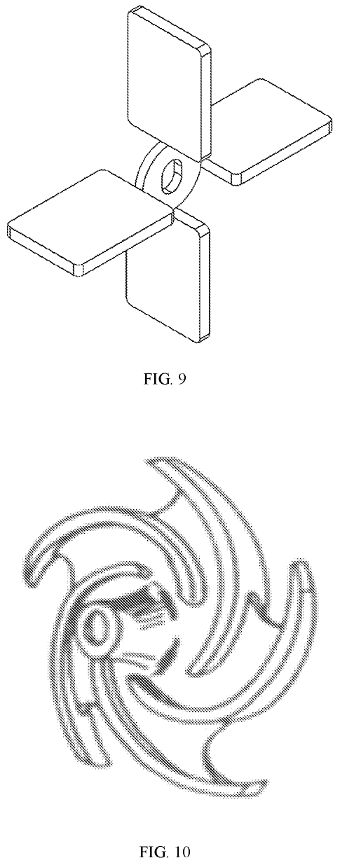

The electrophoresis results were shown in FIG. 8, and it can be seen from FIG. 8 that the plasmid DNA obtained by the method using the bubble generator in the neutralization process of the present comparative example had comparable plasmid concentration as the plasmid DNA obtained by the method of Example 1, but had more host RNAs, indicating that the preparation method of the present application was better, was easier for scale-up, and was simple to operate.

### Comparative Example 2

Unlike Example 1, the impeller of the centrifugal pump head used in the second mixing pump of this example was as shown in FIG. 9, and the rest were the same.

### Detection by a Microplate Reader:

After neutralization, 80 L of the neutralization reaction solution was obtained, 66 L of a supernatant was obtained by centrifugation in total, the plasmid concentration of the supernatant was measured to be 106.6 mg/L (determined by HPLC), and the lysis yield was 64.5%. The lysis yield was lower than that in Example 1.

### Comparative Example 3

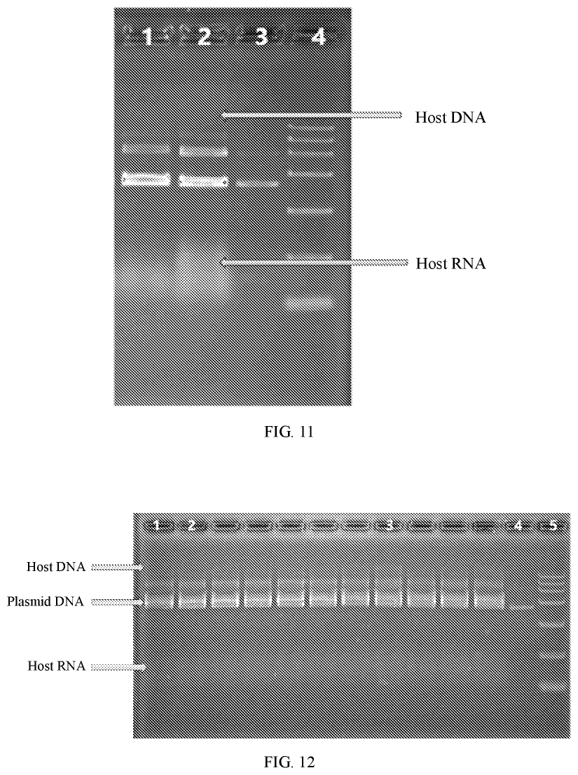

Unlike Example 1, the impeller of the centrifugal pump head used in the second mixing pump of this example was as shown in FIG. 10, and the rest of settings were the same. The electrophoretogram was shown in FIG. 11. It can be seen from FIG. 11 that the use of the pump head shown in FIG. 10 resulted in a larger content of host DNA and RNA in the lysed supernatant, which was not favorable for plasmid purification, compared with the results of Example 1.

Based on the results of the above examples, in the extraction device and the extraction method of the present disclosure, mixing is sufficient and the mixing time is short during lysis of the final product, the neutralization condition is mild and uniform, the residual host DNA and RNA are lower than that of a foaming mixer after the lysis and neutralization, the product quality is good, impurities of extracted plasmid DNA at a moderate speed are fewer, and the yield is high.

The mixing assembly (which can be a pump) is innovatively adopted in the alkaline lysis and neutralization step in the plasmid production process of the present disclosure, so that the lysis and neutralization steps are performed in an airtight environment, the probability of environmental pollution is reduced, CIP and SIP can be conveniently performed after use, continuous processing is realized, the production efficiency is improved, the cost is low; professional customized and expensive equipment is not needed, the scale-up in production is easy, and the production cost is low; the mixing is sufficient during lysis, the mixing time is short, the neutralization condition is mild and uniform, the residual host DNA and RNA are lower than that of a foaming mixer after the lysis and neutralization, and the product quality is good; meanwhile, the size of the pump cavity is optimized, so that the time and the shear force for the lysis and neutralization are suitable for product production, the production scale is convenient to be scaled-up, is easily scaled-up compared with the production system of the current mainstream bubble mixer Airmix, the bubble mixers with different sizes are not required to be customized according to the scale, the exploration time of the scale-up condition is shortened, and the working efficiency is improved.
(2) In the method, the equipment used is simple, the operation is convenient, the two mixing assemblies used can fully mix the bacterium solution and the lysate and ensure that the neutralization solution is mildly mixed and neutralized, the complex low-shear neutralization equipment is avoided, the superhelix proportion of the plasmid after lysis is high, and the residual host DNA and RNA are low; in addition, by using a complex multi-level membrane filtration system, overnight precipitation and other steps are not needed after lysis, the equipment can be directly cleaned by CIP so as to meet the production specification of drug production, meanwhile, the process time is saved, and the cost is reduced; a complex multi-level membrane filtration system is not used, overnight precipitation and other steps are not needed after lysis, the superhelix proportion of the plasmid after lysis is high, the residual host DNA and RNA are low; the equipment can be directly cleaned by CIP so as to meet the production specification of drug production, meanwhile, the process time is saved, the cost is reduced, the operation is convenient, professional customized and expensive equipment is not needed, the scale-up in production is easy, and the production cost is low.
(3) In the process, high-risk animal source components such as RNase, lysozyme, proteinase K and the like are not added, toxic organic solvents such as isopropanol, phenol, absolute ethyl alcohol, other mutagens and the like are not used in the production process, the reagents used can be common reagents or of the medicinal grade, acid liquor is not used for neutralization, the requirement on plant equipment is low, and the method is suitable for large-scale production.
(4) By optimizing the size of the pump cavity and adjusting the ratio of the pump cavity to the flow rate, the time and shear force for the lysis and neutralization are suitable for product production, and meanwhile, the scale-up of production is also facilitated; by optimizing the property and size of the mixing pump head, using 3D printing technique, and designing and customizing the pump head and in the case that the mixing effect is ensured, the shear force is reduced, host DNA is prevented from polluting products, and lysis and neutralization can be performed automatically.

The above description is only for the purpose of illustrating the preferred examples of the present disclosure and is not intended to limit the present disclosure. Any modification, equivalent substitution, improvement and the like made without departing from the spirit and principle of the present disclosure shall fall within the protection scope of the present disclosure.

1. A method for extracting plasmid DNA from bacteria, wherein lysis and neutralization in plasmid DNA production process are implemented in two mixing assemblies connected in series, specifically comprising the following steps:
(1) mixing;
(2) lysing;
(3) neutralizing;
wherein step (1) is completed in a first mixing assembly, step (2) is completed in a spiral lysis pipe, step (3) is completed in a second mixing assembly, and the first mixing assembly, the spiral lysis pipe, and the second mixing assembly are sequentially connected in series.

2. The method of claim 1, wherein the first mixing assembly has a rotational speed of 50 rpm to 1,500 rpm, preferably 200 rpm to 500 rpm; the second mixing assembly has a rotational speed of 20 rpm to 1,000 rpm, preferably 150 rpm to 500 rpm.

3. The method of claim 1, wherein the structures of the first mixing assembly and the second mixing assembly are each independently selected from any one of a stirring type, an emulsifying type and a centrifugal type, and the first mixing assembly and the second mixing assembly are both mixing pumps or stirrers.

4. The method of claim 3, wherein the first mixing assembly and the second mixing assembly are a first mixing pump and a second mixing pump respectively.

5. The method of claim 1, wherein the spiral lysis pipe has an inner diameter of 0.5 cm to 15 cm, preferably 0.5 cm to 6 cm.

6. The method of claim 1, wherein in step (2), the lysis time is 2 min to 10 min, preferably 5 min.

7. The method of claim 4, wherein impellers of the first mixing pump and the second mixing pump both comprise a rear cover plate; a plurality of flow guide columns are uniformly distributed on the rear cover plate, and the outer side surface at least along the rotating direction of the impeller on the flow guide column is arranged as an arc surface.

8. The method for of claim 7, wherein the flow guide column is a cylinder, a circular truncated cone, a fan-shaped column, or a combination of one or more thereof.

9. The method of claim 1, specifically comprising the following steps:
(1) resuspending the bacteria by using the solution I to obtain a resuspended bacterium solution, and then introducing the resuspended bacterium solution and solution II into the first mixing assembly for mixing to obtain a bacteria mixed solution;
(2) the bacteria mixed solution flowing out of the first mixing assembly and entering the spiral lysis pipe for lysis, to obtain a lysate after lysis;
(3) introducing the lysate and solution III into the second mixing assembly (or mixing the lysate and the solution III before introducing them into the second mixing assembly) and then performing a neutralization reaction to obtain a neutralization reaction solution after the neutralization reaction is finished.

10. The method of claim 9, wherein in step (1), the volume to mass ratio of the solution I to the bacteria is 3-20:1 (L:kg), preferably 7:1 (L:kg), the volume ratio of the solution I to the solution II is 1:0.5-3, preferably 1:1; or
in step (3), the volume ratio of the lysate to the solution III is 1:0.3-5, preferably 1:1.

11. The method of claim 3, wherein the first mixing assembly is of the stirring type or the emulsifying type or the centrifugal type, and the second mixing assembly is of the centrifugal type.

12. The method of claim 4, wherein the ratios of the pump cavity volume of the first mixing pump and the pump cavity volume of the second mixing pump to the rated feed volume per minute of a single mixing pump are both in the range of 1:6-1:1, preferably 1:6-1:3.

13. The method of claim 4, wherein the volumes of the pump cavities of the first mixing pump and the second mixing pump are both the volume of the feed liquid flowing through the pump cavity for 10 s to 60 s, preferably the volume of the feed liquid flowing through the pump cavity for 10 s to 20 s.

14. The method of claim 4, wherein pump heads of the first mixing pump and the second mixing pump both have a diameter of 2 cm to 100 cm, preferably 4 cm to 30 cm.

15. The method of claim 8, wherein the cross section of the flow guide column has a width of 0.5 mm to 40 mm, preferably 2 mm to 10 mm.

16. The method of claim 8, wherein the flow guide column is a cylinder.

17. The method of claim 8, wherein the cross-sectional area of the middle of the flow guide column is the largest, and the cross-sectional areas from the middle to the two ends gradually decrease.

18. The method of claim 9, wherein after obtaining the neutralization reaction solution, the method further comprising a step of performing solid-liquid separation and purification on the neutralization reaction solution.

19. The method of claim 18, wherein the solid-liquid separation is carried out by a filtration assembly, wherein the structure of the filtration assembly is a sieve type, a depth filtration type, a centrifugal filtration type, or a combination of one or more thereof.

20. The method of claim 19, wherein the filtration assembly has a structure of a sieve or depth filtration type; the pore size of the filter is 0.2 μm to 800 μm; the filter material includes cellulose, diatomite, activated carbon, polypropylene fiber or silica gel.
